SEQUENCE LISTING (28 sequences)

SEQ ID NO: 1 - protein, 23 aa, sheep
QAVLTQPSSVSRSLGQTVSITCS

SEQ ID NO: 2 - protein, 13 aa, sheep
WFQLIPGSAPKTL

SEQ ID NO: 3 - protein, 31 aa, sheep
VPDRFSGSRSGNTATLTINSLQAEDEADYYC

SEQ ID NO: 4 - protein, 13 aa, sheep
SGTRLTVLSQPKS

SEQ ID NO: 5 - protein, 25 aa, sheep
QVRLQESGPSQVKPAQTLSLTCAIS

SEQ ID NO: 6 - protein, 15 aa, sheep
WVRHTPGKPPEWLAQ

SEQ ID NO: 7 - protein, 30 aa, sheep
RLSITRDTSKSQVFLKLTGVTTEDTAVYYC

SEQ ID NO: 8 - protein, 9 aa, sheep
PGLLVTVSS

SEQ ID NO: 9 - protein, 101 aa, sheep
APSVTLFPPSKEELDTNKATVVCLISDFYPGSVNVVWKADGSTINQNVKTTQASKQSNSKYAASSYLTLTGSEWKSKSSYTCEVTHEGSTVTKTVKPSECS

SEQ ID NO: 10 - protein, 331 aa, sheep
ASTTPPKVYPLTSCCGDTSSSIVTLGCLVSSYMPEPVTVTWNSGALTSGVHTFPAILQSSGLYSLSSVVTVPASTSGAQTFICNVAHPASSTKVDKRVEPGCPDPCKHCRCPPPELPGGPSVFIFPPKPKDTLTISGTPEVTCVVVDVGQDDPEVQFSWFVDNVEVRTARTKPREEQFNSTFRVVSALPIQHQDWTGGKEFKCKVHNEGLPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQKSISKPPGK

SEQ ID NO: 11 - protein, 216 aa, Artificial Sequence
  light chain
QAVLTQPSSVSRSLGQTVSITCSGSSDQVGFGDYINWFQLIPGSAPKTLIFGAYSRIRGVPDRFSGSRSGNTATLTINSLQAEDEADYYCGSPNSRGGYVFGSGTRLTVLSQPKSAPSVTLFPPSKEELDTNKATVVCLISDFYPGSVNVVWKADGSTINQNVKTTQASKQSNSKYAASSYLTLTGSEWKSKSSYTCEVTHEGSTVTKTVKPSECS

SEQ ID NO: 12 - protein, 443 aa, Artificial Sequence
  heavy chain
QVRLQESGPSQVKPAQTLSLTCAISGFSITTYYLDWVRHTPGKPPEWLAQMTKDGNAVHQPTVRSRLSITRDTSKSQVFLKLTGVTTEDTAVYYCVRGVIDFGPGLLVTVSSASTTPPKVYPLTSCCGDTSSSIVTLGCLVSSYMPEPVTVTWNSGALTSGVHTFPAILQSSGLYSLSSVVTVPASTSGAQTFICNVAHPASSTKVDKRVEPGCPDPCKHCRCPPPELPGGPSVFIFPPKPKDTLTISGTPEVTCVVVDVGQDDPEVQFSWFVDNVEVRTARTKPREEQFNSTFRVVSALPIQHQDWTGGKEFKCKVHNEGLPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQKSISKPPGK

SEQ ID NO: 13 - protein, 10 aa, Artificial Sequence
  CDR-VH1
  MISC_FEATURE (2)..(2): Xaa is P or F
  MISC_FEATURE (4)..(4): Xaa is I, V or L
  MISC_FEATURE (9)..(9): Xaa is I, V or L
GXSXTTYYXD

SEQ ID NO: 14 - protein, 15 aa, Artificial Sequence
  CDR-VH2
  MISC_FEATURE (5)..(5): Xaa is G or A
  MISC_FEATURE (10)..(10): Xaa is Q or N
  MISC_FEATURE (13)..(13): Xaa is I, V or L
MTKDXNAVHXPTXRS

SEQ ID NO: 15 - protein, 8 aa, Artificial Sequence
  CDR-VH3
  MISC_FEATURE (2)..(2): Xaa is K or R
  MISC_FEATURE (4)..(4): Xaa is  I, V or L
  MISC_FEATURE (7)..(7): Xaa is  F or W
VXGXIDXG

SEQ ID NO: 16 - protein, 13 aa, Artificial Sequence
  CDR-VL1
  MISC_FEATURE (5)..(5): Xaa is Q or N
  MISC_FEATURE (8)..(8): Xaa is F or P
  MISC_FEATURE (12)..(12): Xaa is I, V or L
GSSDXVGXGDYXN

SEQ ID NO: 17 - protein, 10 aa, Artificial Sequence
  CDR-VL2
  MISC_FEATURE (3)..(3): Xaa is A or G
  MISC_FEATURE (5)..(5): Xaa is T, Y or S
  MISC_FEATURE (8)..(8): Xaa is  I, V or L
IFXAXSRXRG

SEQ ID NO: 18 - protein, 11 aa, Artificial Sequence
  CDR-VL3
  MISC_FEATURE (3)..(3): Xaa is P, A or G
  MISC_FEATURE (7)..(7): Xaa is GG or N
  MISC_FEATURE (10)..(10): Xaa is W or F
GSXNSRXYVXG

SEQ ID NO: 19 - DNA, 25 bp, Artificial Sequence
  SMARTER II A oligonucleotide from a commerical kit
  misc_feature (25)..(25): XXXXX at the 3′-end
aagcagtggtatcaacgcagagtac

SEQ ID NO: 20 - DNA, 27 bp, Artificial Sequence
  5′-RACE CDS primer
  misc_feature (26)..(26): V=A, G, or C
  misc_feature (27)..(27): N=A, C, G, or T
tttttttttttttttttttttttttvn

SEQ ID NO: 21 - DNA, 45 bp, Artificial Sequence
  universal primer A mixture
ctaatacgactcactatagggcaagcagtggtatcaacgcagagt

SEQ ID NO: 22 - DNA, 23 bp, Artificial Sequence
  nested universal primer A
aagcagtggtatcaacgcagagt

SEQ ID NO: 23 - DNA, 35 bp, Artificial Sequence
  mkR
cccgaattcctaagaacactctgagggcttcactg

SEQ ID NO: 24 - DNA, 33 bp, Artificial Sequence
  mHR
cccgaattctcatttacccggaggcttagagat

SEQ ID NO: 25 - DNA, 42 bp, Artificial Sequence
  NT-proBNP 8B9-HF
cccaagcttgccaccatgaacccactgtggaccctcctcttt

SEQ ID NO: 26 - DNA, 33 bp, Artificial Sequence; identical to SEQ ID NO: 24
  NT-proBNP 8B9-HR

SEQ ID NO: 27 - DNA, 41 bp, Artificial Sequence
  NT-proBNP 8B9-LF
cccaagcttgccaccatggcctggtcccctctgctcctcac

SEQ ID NO: 28 - DNA, 35 bp, Artificial Sequence; identical to SEQ ID NO: 23
  NT-proBNP 8B9-LR

(19) United States
(12) Patent Application Publication
(10) Pub. No.: US 2022/0025008 A1
(43) Pub. Date: Jan. 27, 2022

(54) RECOMBINANT ANTIBODY OF ANTI-HUMAN N-TERMINAL PRO-BRAIN NATRIURETIC PEPTIDE

(71) Applicant: Fapon Biotech Inc., Shenzhen,Guangdong (CN)

(72) Inventors: Yuan MENG, Dongguan, Guangdong (CN); Dongmei ZHONG, Dongguan, Guangdong (CN); Qingni YE, Dongguan, Guangdong (CN); Bi LIANG, Dongguan, Guangdong (CN); Hui YOU, Dongguan, Guangdong (CN); Qiuyan MA, Dongguan, Guangdong (CN); Weizhi LI, Dongguan, Guangdong (CN)

(21) Appl. No.: 17/293,044

(22) PCT Filed: Oct. 1, 2019
(86) PCT No.: PCT/CN2019/109792

§ 371 Date: May 12, 2021

(30) Foreign Application Priority Data
Dec. 19, 2018 (CN) 201811557468.5

(51) Int. Cl. C07K 14/575

(52) U.S. Cl. CPC C07K 14/575; C07K 2317/565; C07K 2317/94; C07K 2317/567; C07K 2317/92; C07K 2317/52

(57) ABSTRACT

The present disclosure relates to a novel isolated binding protein including a N-terminal pro-brain natriuretic peptide (NT-proBNP) antigen binding domain, and the preparation method therefor. The antigen-binding domain includes at least one complementarity determining region selected from the amino acid sequences as defined in the present disclosure: or; has at least 80% sequence identity with the complementarity determining region of the following amino acid sequence and has an affinity of KD≤2.26×10⁻⁸ to NT-proBNP. The binding protein may be used in the detection field of NT-proBNP protein.

### CROSS-REFERENCE TO RELATED APPLICATION

The present application is a National Stage of International Patent Application No: PCT/CN2019/109792 filed on Oct. 1, 2019, which claims the benefit of the priority of the Chinese patent application with the application No. 201811557468.5, titled “Recombinant Antibody of Anti-human N-terminal Pro-brain Natriuretic Peptide” filed to the China National Intellectual Property Administration on Dec. 19, 2018, the entire content of which is incorporated in this application by reference.

### SEQUENCE LISTING

The present application contains a Sequence Listing which has been submitted electronically in ASCII format and is hereby incorporated by reference in its entirety. Said ASCII copy is named_Sequence_Listing.txt and is 16.0 kilobytes in size, and contains 16 new sequences from SEQ ID NO:13 to SEQ ID NO:28 described in claims and examples of this file, but not numbered. The original sequences of SEQ ID NO: 1 to SEQ ID NO:12 are identical to the sequence listing filed in the corresponding international application No: PCT/CN2019/109792 filed on Oct. 1, 2019.

### TECHNICAL FIELD

The present disclosure relates to the field of immune technology, in particular, to a recombinant antibody of anti-human N-terminal pro-brain natriuretic peptide.

### BACKGROUND

In 1988, Japanese scholar Sudoh first isolated a polypeptide with potent diuretic, vasodilator and hypotensive effects from pig brain, and named it Brain Natriuretic Peptide (BNP). BNP is most distributed in the heart, but cardiomyocytes first synthesize proBNP containing 108 amino acids. When cardiomyocytes are stimulated, proBNP is cleaved into non-biologically active N-terminal pro-B-type natriuretic peptide (NT-proBNP) containing 76 amino acids and active B-type natriuretic peptide (BNP) containing 32 amino acids, both of which are derived from the same source and are secreted equimolarly and released Into the blood circulation.

When the heart volume load increases or the cardiac function is impaired, the index concentrations of N-terminal pro-brain natriuretic peptide (NT-proBNP) and BNP will Increase abnormally, wherein NT-proBNP has better biological stability, has a longer half-life (120 min), has relatively stable concentration, has a long effective detection time, and has about 16-20 times higher content in blood compared with BNP, therefore, it is relatively easy to detect, and the stability of its plasma samples in vitro is long (>48 h), which is the best myocardial marker for diagnosing heart failure and evaluating cardiac function.

The content of NT-proBNP in the blood of normal people is generally less than 0.3 ng/mL. When the heart function is impaired and the myocardium expands, NT-proBNP will be rapidly synthesized and secreted in large quantities into the human blood. When finding some relevant early symptoms, accurate, sensitive, efficient and stable determination of the amount of NT-proBNP In the blood can provide a fast and accurate basis for early diagnosis for early cardiac insufficiency, heart failure, cardiogenic and non-cardiogenic heart failure with dyspnea treatment and prognosis monitoring, and acute coronary syndrome grading, etc. The current methods used to detect the content of NT-proBNP mainly include gold calibration test, fluorescence immunoassay, enzyme-inked immunosorbent assay (ELISA) and chemiluminescence microparticle immunoassays (CMIA), but these measurement methods all require a specific monoclonal antibody against NT-proBNP. However, the sensitivity and specificity of the monoclonal antibody currently used to detect NT-proBNP in China are not ideal.

### SUMMARY

The present disclosure relates to a novel isolated binding protein including a N-terminal pro-brain natriuretic peptide (NT-proBNP) antigen binding domain, and investigates the preparation, use and other aspects of the binding protein.

Wherein the antigen-binding domain includes at least one complementarity determining region (CDR) selected from the following amino acid sequences: or has at least 80% sequence identity with the complementarity determining region of the following amino acid sequence and has an affinity of K_{D}≤2.26×10⁻⁸ to NT-proBNP:

the complementarity determining region CDR-VH1 is G-X1-S-X2-T-T-Y-Y-X3-D (SEQ ID NO:13), wherein

X1 is P or F, X2 is I, V or L, and X3 is I, V or L;

the complementarity determining region CDR-VH2 is M-T-K-D-X1-N-A-V-H-X2-P-T-X3-R-S (SEQ ID NO:14), wherein

X1 is G or A, X2 is Q or N, and X3 is I, V or L;

the complementarity determining region CDR-VH3 is V-X1-G-X2-1-D-X3-G (SEQ ID NO:15), wherein

X1 is K or R, X2 is I, V or L, and X3 is F or W;

the complementarity determining region CDR-VL1 is G-S-S-D-X1-V-G-X2-G-D-Y-X3-N (SEQ ID NO:16), wherein

X1 is Q or N, X2 is F or P, and X3 is I, V or L;

the complementarity determining region CDR-VL2 is I-F-X1-A-X2-S-R-X3-R-G (SEQ ID NO:17), wherein

X1 is A or G, X2 is T, Y or S, and X3 is 1, V or L;

the complementarity determining region CDR-VL3 is G-S-X1-N-S-R-X2-Y-V-X3-G (SEQ ID NO:18), wherein

X1 is P, A or G, X2 is GG or N, and X3 is W or F.

The binding protein has an Important advantage in that it has strong activity and high affinity to human NT-proBNP.

In one or more embodiments:

X1 is F in the complementarity determining region CDR-VH1;

X1 is G in the complementarity determining region CDR-VH2;

X1 is R in the complementarity determining region CDR-VH3;

X2 is F in the complementarity determining region CDR-VL1;

X1 is G in the complementarity determining region CDR-VL2;

X3 is F in the complementarity determining region CDR-VL3.

In one or more embodiments, X2 is I in the complementarity determining region CDR-VH1.

In one or more embodiments, X2 is V in the complementarity determining region CDR-VH1.

In one or more embodiments, X2 is L in the complementarity determining region CDR-VH1.

In one or more embodiments, X3 is I in the complementarity determining region CDR-VH1.

In one or more embodiments, X3 is V in the complementarity determining region CDR-VH1.

In one or more embodiments, X3 is L in the complementarity determining region CDR-VH1.

In one or more embodiments, X2 is Q in the complementarity determining region CDR-VH2.

In one or more embodiments, X2 is N in the complementarity determining region CDR-VH2.

In one or more embodiments, X3 is I in the complementarity determining region CDR-VH2.

In one or more embodiments, X3 is V in the complementarity determining region CDR-VH2.

In one or more embodiments, X3 is L in the complementarity determining region CDR-VH2.

In one or more embodiments, X2 is I in the complementarity determining region CDR-VH3.

In one or more embodiments, X2 is V In the complementarity determining region CDR-VH3.

In one or more embodiments, X2 is L in the complementarity determining region CDR-VH3.

In one or more embodiments, X3 is F in the complementarity determining region CDR-VH3.

In one or more embodiments, X3 is W in the complementarity determining region CDR-VH3.

In one or more embodiments, X1 is Q in the complementarity determining region CDR-VL1.

In one or more embodiments, X1 is N in the complementarity determining region CDR-VL1.

In one or more embodiments, X3 is I in the complementarity determining region CDR-VL1.

In one or more embodiments, X3 is V in the complementarity determining region CDR-VL1.

In one or more embodiments, X3 is L in the complementarity determining region CDR-VL1.

In one or more embodiments, X2 is T in the complementarity determining region CDR-VL2.

In one or more embodiments, X2 is Y in the complementarity determining region CDR-VL2.

In one or more embodiments, X2 is S in the complementarity determining region CDR-VL2.

In one or more embodiments, X3 is I in the complementarity determining region CDR-VL2.

In one or more embodiments, X3 is V in the complementarity determining region CDR-VL2.

In one or more embodiments, X3 is L in the complementarity determining region CDR-VL2.

In one or more embodiments, X1 is P in the complementarity determining region CDR-VL3.

In one or more embodiments, X1 is A in the complementarity determining region CDR-VL3.

In one or more embodiments, X1 is G in the complementarity determining region CDR-VL3.

In one or more embodiments, X2 is GG in the complementarity determining region CDR-VL3.

In one or more embodiments, X2 is N in the complementarity determining region CDR-VL3.

In one or more embodiments, the mutation site of each complementarity determining region is selected from any one of the following mutation combinations:

| | CDR-VH1 | CDR-VH2 | CDR-VH3 | CDR-VL1 | CDR-VL2 | CDR-VL3 |
|---|---|---|---|---|---|---|
| Site | X2/X3 | X2/X3 | X2/X3 | X1/X3 | X2/X3 | X1/X2 |
| Mutation combination 1 | I/I | Q/I | I/W | Q/V | T/I | P/GG |
| Mutation combination 2 | I/L | N/I | I/F | Q/L | T/L | P/N |
| Mutation combination 3 | I/V | Q/L | L/W | Q/I | T/V | A/GG |
| Mutation combination 4 | L/I | N/L | L/F | N/V | V/I | A/N |
| Mutation combination 5 | L/L | Q/V | V/W | N/L | Y/L | G/GG |
| Mutation combination 6 | L/V | N/V | V/F | N/I | Y/V | G/N |
| Mutation combination 7 | V/I | N/V | I/W | N/I | S/I | P/N |
| Mutation combination 8 | V/L | Q/V | I/F | N/L | S/L | G/N |
| Mutation combination 9 | V/V | N/L | L/W | N/V | S/V | G/GG |
| Mutation combination 10 | I/I | Q/L | L/F | Q/I | S/I | A/GG |
| Mutation combination 11 | I/L | N/I | V/W | Q/L | S/L | A/N |
| Mutation combination 12 | I/V | Q/I | V/F | Q/V | S/V | P/GG |
| Mutation combination 13 | L/I | Q/I | I/W | Q/V | V/I | P/GG |
| Mutation combination 14 | L/L | N/I | I/F | Q/L | Y/L | P/N |
| Mutation combination 15 | L/V | Q/L | L/W | Q/I | Y/V | A/GG |
| Mutation combination 16 | V/I | N/L | L/F | N/V | T/I | A/N |
| Mutation combination 17 | V/L | Q/V | V/W | N/L | T/L | G/GG |
| Mutation combination 18 | V/V | N/V | V/F | N/I | T/V | G/N |
| Mutation combination 19 | I/I | N/V | I/W | N/I | T/I | P/N |
| Mutation combination 20 | I/L | Q/V | I/F | N/L | T/L | G/N |
| Mutation combination 21 | I/V | N/L | L/W | N/V | T/V | G/GG |
| Mutation combination 22 | L/I | Q/L | L/F | Q/I | V/I | A/GG |
| Mutation combination 23 | L/L | N/I | V/W | Q/L | Y/L | A/N |
| Mutation combination 24 | L/V | Q/I | V/F | Q/V | Y/V | P/GG |
| Mutation combination 25 | V/I | Q/I | I/W | Q/V | S/I | P/GG |
| Mutation combination 26 | V/L | N/I | I/F | Q/L | S/L | P/N |
| Mutation combination 27 | V/V | Q/L | L/W | Q/V | S/V | A/GG |
| Mutation combination 28 | I/I | N/L | L/F | N/V | S/I | A/N |
| Mutation combination 29 | I/L | Q/V | V/W | N/L | S/L | G/GG |
| Mutation combination 30 | I/V | N/V | V/F | N/I | S/V | G/N |
| Mutation combination 31 | L/I | N/V | I/W | N/I | T/I | P/N |
| Mutation combination 32 | L/L | Q/V | I/F | N/L | T/L | G/N |
| Mutation combination 33 | L/V | N/L | L/W | N/V | T/V | G/GG |
| Mutation combination 34 | V/I | Q/L | L/F | Q/I | Y/I | A/GG |
| Mutation combination 35 | V/L | N/I | V/W | Q/L | Y/L | A/N |
| Mutation combination 36 | V/V | Q/I | V/F | Q/V | Y/V | P/GG |
| Mutation combination 37 | I/I | Q/I | I/W | Q/V | T/I | P/GG |
| Mutation combination 38 | I/L | N/I | I/F | Q/L | T/L | A/N |
| Mutation combination 39 | I/V | Q/L | L/W | Q/I | T/V | G/GG |
| Mutation combination 40 | L/I | N/L | L/F | N/V | Y/I | G/N |
| Mutation combination 41 | L/L | Q/V | V/W | N/L | Y/L | P/N |
| Mutation combination 42 | V/I | N/V | I/W | N/I | Y/V | G/N |
| Mutation combination 43 | V/I | N/V | I/W | N/I | S/I | G/GG |
| Mutation combination 44 | V/L | Q/V | I/F | N/L | S/L | A/GG |
| Mutation combination 45 | V/V | N/L | L/W | N/V | S/V | A/N |
| Mutation combination 46 | I/I | Q/L | L/F | Q/I | S/I | P/GG |
| Mutation combination 47 | I/L | N/I | V/W | Q/L | S/L | P/GG |
| Mutation combination 48 | I/V | Q/I | V/F | Q/V | S/V | P/N |
| Mutation combination 49 | L/I | Q/I | I/W | Q/V | T/I | P/GG |
| Mutation combination 50 | L/L | N/I | I/F | Q/L | T/L | P/N |
| Mutation combination 51 | L/V | Q/L | L/W | Q/I | T/V | A/GG |
| Mutation combination 52 | V/I | N/L | L/F | N/V | Y/I | A/N |
| Mutation combination 53 | V/L | Q/V | V/W | N/L | Y/L | G/GG |
| Mutation combination 54 | V/V | N/V | V/F | N/I | Y/V | G/N. |

In one or more embodiments, the binding protein includes at least 3 CDRs (for example, 3 light chain CDRs or 3 heavy chain CDRs); alternatively, the binding protein includes at least 6 CDRs.

In one or more embodiments, the binding protein is an intact antibody including a variable region and constant region.

In one or more embodiments, the binding protein includes light chain framework regions FR-1, FR-L2, FR-L3 and FR-L4 with sequence correspondingly shown in SEQ ID NO: 1-4, and/or, heavy chain framework regions FR-1, FR-H2, FR-H3 and FR-H4 with sequence correspondingly shown in SEQ ID NO: 5-8.

In one or more embodiments, the binding protein further includes an antibody constant region sequence.

In one or more embodiments, the constant region sequence is a sequence of any one constant region selected from IgG1, IgG2, IgG3, IgG4, IgA, IgM, IgE, and IgD.

In one or more embodiments, the constant region is derived from species consisted of cattle, horse, dairy cow, pig, sheep, goat, rat, mouse, dog, cat, rabbi, camel, donkey, deer, mink, chicken, duck, goose, turkey, gamecock or human.

In one or more embodiments, the constant region is derived from the sheep;

a light chain constant region sequence is shown in SEQ ID NO: 9;

a heavy chain constant region sequence is shown in SEQ ID NO: 10.

The present disclosure also relates to an isolated nucleic acid molecule, encoding the above binding protein.

The present disclosure also provides a vector, including the above nucleic acid molecule.

The present disclosure also relates to a host cell transformed with the above vector.

The present disclosure also relates to a method for producing the above binding protein, the method including the steps of:

culturing the above host cell in a culture medium and under suitable culture conditions, recovering such produced binding protein from the culture medium or from the cultured host cell.

According to an aspect of the present disclosure, the present disclosure also relates to use of the above binding protein in preparation of a diagnostic agent for diagnosing heart failure and evaluating a cardiac function.

According to an aspect of the present disclosure, the present disclosure also relates to a method for detecting NT-proBNP in a test sample, the method including:

a) contacting the NT-proBNP antigen in the test sample with the above binding protein to form an immune complex under conditions sufficient for taking an antibody/antigen binding reaction; and

b) detecting a presence of the immune complex, which indicates a presence of the NT-proBNP in the test sample.

In one or more embodiments, in step a), the immune complex further includes a second antibody that binds to the binding protein;

In one or more embodiments, in step a), the immune complex further includes a second antibody that binds to the NT-proBNP;

According to an aspect of the present disclosure, the present disclosure further relates to a kit, including the above binding protein.

The present disclosure further relates to use of the binding protein described herein in diagnosing a disease related to NT-proBNP.

The present disclosure further relates to a method for diagnosing a disease related to NT-proBNP or evaluating a cardiac function, the method including:

A) contacting a sample from a subject with the binding protein described in the present disclosure for a binding reaction under conditions sufficient for taking a binding reaction; and

B) detecting the immune complex produced by the binding reaction,

wherein the presence of the immune complex indicates the presence of a disease related to NT-proBNP or indicates the level of the cardiac function.

In one or more embodiments, the method is based on fluorescence immunoassay, chemiluminescence, colloidal gold immunoassay, radioimmunoassay, and/or enzyme-linked immunoassay.

In one or more embodiments, the sample is selected from at least one of whole blood, peripheral blood, serum, plasma or myocardial tissue.

In one or more embodiments, the subject is mammal, preferably primate, more preferably human.

In one or more embodiments, the disease related to NT-proBNP is a cardiac disease.

In one or more embodiments, the disease related to NT-proBNP is selected from heart failure, cardiac insufficiency, cardiogenic dyspnea, pulmonary dyspnea, acute coronary syndrome, or a combination thereof.

In one or more embodiments, the heart failure is cardiogenic heart failure or non-cardiac heart failure.

### BRIEF DESCRIPTION OF THE DRAWINGS

In order to more clearly illustrate the specific embodiments of the present disclosure or the technical solutions in the prior art, specific embodiments or drawings that may be required in prior art descriptions are briefly described below, obviously, the drawings described below are some embodiments of the present disclosure. For those of ordinary skill in the art, other drawings can be obtained based on these drawings without creative work.

FIG. 1 is an electropherogram of a monoclonal antibody of the anti-human NT-proBNP in an embodiment of the present disclosure.

### DETAILED DESCRIPTION OF THE EMBODIMENTS

The present disclosure can be more easily understood through the following description of some embodiments of the present disclosure and the detailed content of the embodiments included therein.

Before further describing the present disclosure, it should be understood that the present disclosure is not limited to the specific embodiments, because these embodiments are necessarily diverse. It should also be understood that the terms used in this specification are only to illustrate specific embodiments, rather than as limitations, because the scope of the present disclosure will only be defined in the appended claims.

### Definition

“Isolated binding protein including an antigen binding domain” broadly refers to all proteins/protein fragments including a CDR region. The term “antibody” includes a polyclonal antibody, a monoclonal antibody, and the antigen compound binding fragments of these antibodies, including Fab, F(ab)₂, Fd, Fv, scFv, a bispecific antibody and a minimum recognition unit of antibody, as well as single-chain derivatives of these antibodies and fragments. The type of antibody can select from IgG1, IgG2, IgG3, IgG4, IgA, IgM, IgE and IgD. In addition, the term “antibody” includes a naturally-occurring antibody and a non-naturally-occurring antibody, including, for example, chimeric, bifunctional, and humanized antibodies, and related synthetic isoforms. The term “antibody” can be used interchangeably with “immunoglobulin”.

A “variable region” or “variable domain” of an antibody refers to an amino terminal domain of a heavy or light chain of an antibody. A variable domain of a heavy chain can be referred to as “VH”. A variable domain of a light chain can be referred to as “VL”. These domains are usually the most variable part of the antibody and contain the antigen binding site. A variable region of a light chain or a heavy chain (VL or VH) is consisted of three called “complementarity determining regions” or “CDRs” and the framework regions that separate the three complementarity determining regions. The scope of the framework region and CDR has been precisely defined, for example in Kabat (see “Sequences of Proteins of Immunological Interest”, E. Kabat, etc., U.S. Department of Health and Human Services, (1983)) and Chothia. The framework region of the antibody plays a role in positioning and aligning the CDRs that are mainly responsible for binding to the antigen.

As used herein, “framework region”, “framework regions” or “FR” means that the excluded antibody variable domains are regions other than those defined as CDRs. Each antibody variable domain framework region can be further subdivided into adjacent regions (FR1, FR2, FR3 and FR4) separated by CDRs.

Generally, the variable regions VL/VH of the heavy chain and the light chain can be obtained by arranging and linking the following numbered CDRs and FRs in the following combination: FR1-CDR1-FR2-CDR2-FR3-CDR3-FR4.

As used herein, the term “purified” or “isolated” associated with a polypeptide or nucleic acid means that the polypeptide or nucleic acid is not in its natural medium or in its natural form. Thus, the term “isolated” includes a polypeptide or nucleic acid taken from its original environment, for example, from the natural environment if it is naturally occurring. For example, an isolated polypeptide generally does not contain at least some proteins or other cellular components that are normally bound to it or usually mixed with it or in solution. An isolated polypeptide includes a naturally produced polypeptide contained in the cell lysate, a polypeptide in purified or partially purified form, a recombinant polypeptide, a polypeptide expressed or secreted by a cell, and a polypeptide in a heterologous host cell or the culture. Associated with nucleic acid, the term isolated or purified indicates that, for example, the nucleic acid is not in its natural genomic context (for example, in a vector, as an expression cassette, linked to a promoter, or artificially introduced into a heterologous host cell).

As used herein, the term “bispecific antibody” or “bifunctional antibody” refers to an artificial hybrid binding protein with two different pairs of heavy/light chains and two different binding sites. The bispecific binding protein can be produced by a variety of methods, including fusion of hybridomas or linking of Fab fragments.

As used herein, the term “sequence identity” refers to the similarity between at least two different sequences. The percentage identity can be determined by standard algorithms, such as Basic Local Alignment Search Tool (BLAST); an algorithm described by Needleman et al.; or an algorithm described by Meyers et al. In one or more embodiments, a set of parameters may be Blosum 62 scoring matrix and gap penalty of 12, gap extension penalty of 4, and frameshift gap penalty of 5. In one or more embodiments, the percentage identity between two amino acid or nucleotide sequences can also be determined using the algorithm described by Meyers and Miller ((1989) CABIOS 4: 11-17), which has been incorporated to the ALIGN program (version 2.0), using the PAM120 weight residue table, gap length penalty of 12, and gap penalty of 4. Percentage identity is usually calculated by comparing sequences having similar length.

As used herein, the term “affinity” refers to the binding strength of an antigen binding domain of a binding protein or antibody to an antigen or antigen epitope. Affinity can be measured by the KD value, the smaller of which, the greater the affinity.

### EXEMPLARY EMBODIMENTS OF THE PRESENT DISCLOSURE

The present disclosure relates to an isolated binding protein including an antigen-binding domain, wherein the antigen-binding domain includes at least one complementarity determining region selected from the following amino acid sequences: or has at least 80% sequence identity with the complementarity determining region of the following amino acid sequences and having an affinity of K_{D}≤2.26×10⁻⁸ to a NT-proBNP;
the complementarity determining region CDR-VH1 is G-X1-S-X2-T-T-Y-Y-X3-D, wherein
X1 is P or F, X2 is I, V or L, and X3 is I, V or L;

the complementarity determining region CDR-VH2 is M-T-K-D-X1-N-A-V-H-X2-P-T-X3-R-S, wherein

X1 is G or A, X2 is Q or N, and X3 is I, V or L;

the complementarity determining region CDR-VH3 is V-X1-G-X2-I-D-X3-G, wherein

X1 is K or R, X2 is I, V or L, and X3 is F or W;

the complementarity determining region CDR-VL1 is G-S-S-D-X1-V-G-X2-G-D-Y-X3-N, wherein

X1 is Q or N, X2 is F or P, and X3 is I, V or L;

the complementarity determining region CDR-VL2 is I-F-X1-A-X2-S-R-X3-R-G, wherein

X1 is A or G, X2 Is T, Y or S, and X3 is I, V or L;

the complementarity determining region CDR-VL3 is G-S-X1-N-S-R-X2-Y-V-X3-G, wherein

X1 is P, A or G, X2 is GG or N, and X3 Is W or F.

As used herein, the terms “N-terminal pro-brain natriuretic peptide (NT-proBNP)” and “N-terminal pro-B-type natriuretic peptide” can be used interchangeably, which refer to the non-biologically active N-terminal fragment produced by pro-brain natriuretic peptide or B-type natriuretic peptide after digestion, for example by endonuclease digestion.

In one or more embodiments, the antigen binding domain has at least 50%, or at least 55%, or at least 60%, or at least 65%, or at least 70%, or at least 75%, or at least 80%, or at least 85%, or at least 90%, or at least 91%, or at least 92%, or at least 93%, or at least 94%, or at least 95%, or at least 96%, or at least 97%, or at least 98%, or at least 99% sequence identity with the complementarity determining region of the following amino acid sequence and having an affinity of K_{D}≤2.26×10⁻⁸ mol/L to NT-proBNP, and the K_{D} value can also select from 1×10⁻⁹ mol/L, 2×10⁻⁹ mol/L, 3×10⁻⁹ mol/L, 4×10⁻⁹ mol/L, 4.5×10⁻⁹ mol/L, 5×10⁻⁹ mol/L, 6×10⁻⁹ mol/L, 7×10⁻⁹ mol/L, 8×10⁻⁹ mol/L, 9×10⁻⁹ mol/L, 1×10⁻¹⁰ mol/L, 3×10⁻¹⁰ mol/L, 5×10⁻¹⁰ mol/L, 7×⁻¹⁰ mol/L, 9×10⁻¹⁰ mol/L or 1×10⁻⁸ mol/L;

or 8.10×10⁻¹⁰ mol/L≤K_{D}≤2.26×10⁻⁸ mol/L;

or KD is less than or equal to 1×10⁻⁹ mol/L, 2×10⁻⁹ mol/L, 3×10⁻⁹ mol/L, 4×10⁻⁹ mol/L, 4.5×10⁻⁹ mol/L, 5×10⁻⁹ mol/L, 6×10⁻⁹ mol/L, 7×10⁻⁹ mol/L, 8×10⁻⁹ mol/L, 9×10⁻⁹ mol/L, 1×10⁻¹⁰ mol/L, 3×10⁻¹⁰ mol/L, 5×10⁻¹⁰ mol/L, 7×10⁻¹⁰ mol/L, 9×10⁻¹⁰ mol/L or 1×10⁻⁸ mol/L;

Wherein, the affinity is measured according to the method in the present disclosure.

In one or more embodiments:

X1 is F in the complementarity determining region CDR-VH1;

X1 is G in the complementarity determining region CDR-VH2;

X1 is R in the complementarity determining region CDR-VH3;

X2 is F in the complementarity determining region CDR-VL1;

X1 is G in the complementarity determining region CDR-VL2;

X3 is F in the complementarity determining region CDR-VL3.

In one or more embodiments, X2 is I in the complementarity determining region CDR-VH1.

In one or more embodiments, X2 is V In the complementarity determining region CDR-VH1.

In one or more embodiments, X2 is L in the complementarity determining region CDR-VH1.

In one or more embodiments, X3 is I in the complementarity determining region CDR-VH1.

In one or more embodiments, X3 is V in the complementarity determining region CDR-VH1.

In one or more embodiments, X3 is L in the complementarity determining region CDR-VH1.

In one or more embodiments, X2 is Q in the complementarity determining region CDR-VH2.

In one or more embodiments, X2 is N in the complementarity determining region CDR-VH2.

In one or more embodiments, X3 is I in the complementarity determining region CDR-VH2.

In one or more embodiments, X3 is V in the complementarity determining region CDR-VH2.

In one or more embodiments, X3 is L in the complementarity determining region CDR-VH2.

In one or more embodiments, X2 is I in the complementarity determining region CDR-VH3.

In one or more embodiments, X2 is V in the complementarity determining region CDR-VH3.

In one or more embodiments, X2 is L in the complementarity determining region CDR-VH3.

In one or more embodiments, X3 is F in the complementarity determining region CDR-VH3.

In one or more embodiments, X3 is W in the complementarity determining region CDR-VH3.

In one or more embodiments, X1 is Q in the complementarity determining region CDR-VL1.

In one or more embodiments, X1 is N in the complementarity determining region CDR-VL1.

In one or more embodiments, X3 is I in the complementarity determining region CDR-VL1.

In one or more embodiments, X3 is V in the complementarity determining region CDR-VL1.

In one or more embodiments, X3 is L in the complementarity determining region CDR-VL1.

In one or more embodiments, X2 is T in the complementarity determining region CDR-VL2.

In one or more embodiments, X2 is Y in the complementarity determining region CDR-VL2.

In one or more embodiments, X2 is S in the complementarity determining region CDR-VL2.

In one or more embodiments, X3 is I in the complementarity determining region CDR-VL2.

In one or more embodiments, X3 is V in the complementarity determining region CDR-VL2.

In one or more embodiments, X3 is L in the complementarity determining region CDR-VL2.

In one or more embodiments, X1 is P in the complementarity determining region CDR-VL3.

In one or more embodiments, X1 is A in the complementarity determining region CDR-VL3.

In one or more embodiments, X1 is G in the complementarity determining region CDR-VL3.

In one or more embodiments, X2 is GG in the complementarity determining region CDR-VL3.

In one or more embodiments, X2 is N in the complementarity determining region CDR-VL3.

In one or more embodiments, the mutation site of each complementarity determining region is selected from any one of the following mutation combinations:

| | CDR-VH1 | CDR-VH2 | CDR-VH3 | CDR-VL1 | CDR-VL2 | CDR-VL3 |
|---|---|---|---|---|---|---|
| Site | X2/X3 | X2/X3 | X2/X3 | X1/X3 | X2/X3 | X1/X2 |
| Mutation combination 1 | I/I | Q/I | I/W | Q/V | T/I | P/GG |
| Mutation combination 2 | I/L | N/I | I/F | Q/L | T/L | P/N |
| Mutation combination 3 | I/V | Q/L | L/W | Q/I | T/V | A/GG |
| Mutation combination 4 | L/I | N/L | L/F | N/V | Y/I | A/N |
| Mutation combination 5 | L/L | Q/V | V/W | N/L | Y/L | G/GG |
| Mutation combination 6 | L/V | N/V | V/F | N/I | Y/V | G/N |
| Mutation combination 7 | V/I | N/V | I/W | N/I | S/I | P/N |
| Mutation combination 8 | V/L | Q/V | I/F | N/L | S/L | G/N |
| Mutation combination 9 | V/V | N/L | L/W | N/V | S/V | G/GG |
| Mutation combination 10 | I/I | Q/L | L/F | Q/I | S/I | A/GG |
| Mutation combination 11 | I/L | N/I | V/W | Q/L | S/L | A/N |
| Mutation combination 12 | I/V | Q/I | V/F | Q/V | S/V | P/GG |
| Mutation combination 13 | L/I | Q/I | I/W | Q/V | Y/I | P/GG |
| Mutation combination 14 | L/L | N/I | I/F | Q/L | Y/L | P/N |
| Mutation combination 15 | L/V | Q/L | L/W | Q/I | Y/V | A/GG |
| Mutation combination 16 | V/I | N/L | L/F | N/V | T/I | A/N |
| Mutation combination 17 | V/L | Q/V | V/W | N/L | T/L | G/GG |
| Mutation combination 18 | V/V | N/V | V/F | N/I | T/V | G/N |
| Mutation combination 19 | I/I | N/V | I/W | N/I | T/I | P/N |
| Mutation combination 20 | I/L | Q/V | I/F | N/L | T/L | G/N |
| Mutation combination 21 | I/V | N/L | L/W | N/V | T/V | G/GG |
| Mutation combination 22 | L/I | Q/L | L/F | Q/I | Y/I | A/GG |
| Mutation combination 23 | L/L | N/I | V/W | Q/L | Y/L | A/N |
| Mutation combination 24 | L/V | Q/I | V/F | Q/V | Y/V | P/GG |
| Mutation combination 25 | V/I | Q/I | I/W | Q/V | S/I | P/GG |
| Mutation combination 26 | V/L | N/I | I/F | Q/L | S/L | P/N |
| Mutation combination 27 | V/V | Q/L | L/W | Q/V | S/V | A/GG |
| Mutation combination 28 | I/I | N/L | L/F | N/V | S/I | A/N |
| Mutation combination 29 | I/L | Q/V | V/W | N/L | S/L | G/GG |
| Mutation combination 30 | I/V | N/V | V/F | N/I | S/V | G/N |
| Mutation combination 31 | L/I | N/V | I/W | N/I | T/I | P/N |
| Mutation combination 32 | L/L | Q/V | I/F | N/L | T/L | G/N |
| Mutation combination 33 | L/V | N/L | L/W | N/V | T/V | G/GG |
| Mutation combination 34 | V/I | Q/L | L/F | Q/I | Y/I | A/GG |
| Mutation combination 35 | V/L | N/I | V/W | Q/L | Y/L | A/N |
| Mutation combination 36 | V/V | Q/I | V/F | Q/V | Y/V | P/GG |
| Mutation combination 37 | I/I | Q/I | I/W | Q/V | T/I | P/GG |
| Mutation combination 38 | I/L | N/I | I/F | Q/L | T/L | A/N |
| Mutation combination 39 | I/V | Q/L | L/W | Q/I | T/V | G/GG |
| Mutation combination 40 | L/I | N/L | L/F | N/V | Y/I | G/N |
| Mutation combination 41 | L/L | Q/V | V/W | N/L | Y/L | P/N |
| Mutation combination 42 | V/I | N/V | I/W | N/I | Y/V | G/N |
| Mutation combination 43 | V/I | N/V | I/W | N/I | S/I | G/GG |
| Mutation combination 44 | V/L | Q/V | I/F | N/L | S/L | A/GG |
| Mutation combination 45 | V/V | N/L | L/W | N/V | S/V | A/N |
| Mutation combination 46 | I/I | Q/L | L/F | Q/I | S/I | P/GG |
| Mutation combination 47 | I/L | N/I | V/W | Q/L | S/L | P/GG |
| Mutation combination 48 | I/V | Q/I | V/F | Q/V | S/V | P/N |
| Mutation combination 49 | L/I | Q/I | I/W | Q/V | T/I | P/GG |
| Mutation combination 50 | L/L | N/I | I/F | Q/L | T/L | P/N |
| Mutation combination 51 | L/V | Q/L | L/W | Q/I | T/V | A/GG |
| Mutation combination 52 | V/I | N/L | L/F | N/V | Y/I | A/N |
| Mutation combination 53 | V/L | Q/V | V/W | N/L | Y/L | G/GG |
| Mutation combination 54 | V/V | N/V | V/F | N/I | Y/V | G/N |

In one or more embodiments, X1s present in the six CDRs of the binding protein described in the present disclosure each independently represent the amino acid defined in the present disclosure; X2s present in the six CDRs of the binding protein described in the present disclosure each independently represent the amino acid defined in the present disclosure; X3s present in the six CDRs of the binding protein described in the present disclosure each independently represent the amino acid defined in the present disclosure.

In one or more embodiments, the binding protein includes at least 3 CDRs (for example, 3 light chain CDRs or 3 heavy chain CDRs); alternatively, the binding protein includes at least 6 CDRs.

In one or more embodiments, the binding protein is an intact antibody including a variable region and constant region.

In one or more embodiments, the binding protein is a “functional fragment” of an antibody, such as a nanobody, F(ab′)₂, Fab′, Fab, Fv, scFv, a bispecific antibody and a minimal recognition unit of antibody.

scFv (sc=single chain), bispecific antibodies (diabodies).

The “functional fragment” described in the present disclosure specifically refers to an antibody fragment having identical specificity as the parent antibody for NT-proBNP. In addition to the above functional fragment, any fragments whose half-life has been Increased are also Included.

These functional fragments usually have identical binding specificity as the antibody from which they are derived. Those skilled in the art infer from the content recorded in the description of the present disclosure that the above functional fragment can be obtained by a method such as enzymatic digestion (including pepsin or papain) and/or a method of splitting a disulfide bond by chemical reduction from the antibody fragment of the present disclosure.

The antibody fragment can also be obtained by peptide synthesis using recombinant genetic techniques that are also known to those skilled in the art or, for example, an automatic peptide synthesizer, such as commercially available from Applied BioSystems.

In one or more embodiments, the binding protein includes light chain framework regions FR-L1, FR-L2, FR-L3 and FR-L4 with sequence correspondingly shown in SEQ ID NO:1-4, and/or, heavy chain framework regions FR-H1, FR-H2, FR-H3 and FR-H4 with sequence correspondingly shown in SEQ ID NO: 5-8.

It should be noted that, in addition to the amino acid sequence disclosed above in this disclosure, the species source of the framework region may be human to constitute a humanized antibody.

In one or more embodiments, the binding protein further includes an antibody constant region sequence.

In one or more embodiments, the constant region sequence is a sequence of any one constant region selected from IgG1, IgG2, IgG3, IgG4, IgA, IgM, IgE, and IgD.

In one or more embodiments, the constant region is derived from species consisted of cattle, horse, dairy cow, pig, sheep, goat, rat, mouse, dog, cat, rabbit, camel, donkey, deer, mink, chicken, duck, goose, turkey, gamecock or human.

In one or more embodiments, the constant region is derived from the sheep; a light chain constant region sequence is shown in SEQ ID NO: 9;

a heavy chain constant region sequence is shown in SEQ ID NO: 10.

According to an aspect of the present disclosure, the present disclosure further relates to an isolated nucleic acid molecule, the nucleic acid molecule being DNA or RNA, which encodes the above binding protein.

According to an aspect of the present disclosure, the present disclosure also relates to a vector, including the above nucleic acid molecular.

The present disclosure further includes at least one nuclear construct encoding the above nucleic acid molecule, such as a plasmid, and further an expression plasmid, a construction method for which will be introduced in an embodiment of the present disclosure.

According to an aspect of the present disclosure, the present disclosure further relates to a host cell transformed with the above vector.

The host cell may be an eukaryotic cell, such as a mammalian cell.

In one or more embodiments, the host cell is a CHO cell.

According to an aspect of the present disclosure, the present disclosure further relates to a method for producing the above binding protein, the method including the steps of: culturing the above host cell in a culture medium and under suitable culture conditions, recovering such produced binding protein from the culture medium or from the cultured host cell.

According to an aspect of the present disclosure, the present disclosure also relates to use of the above binding protein in preparation of a diagnostic agent for diagnosing heart failure and evaluating a cardiac function.

According to one aspect of the present disclosure, the present disclosure further relates to a method for detecting NT-proBNP in a test sample, the method including:

a) contacting the NT-proBNP antigen in the test sample with the above binding protein to form an immune complex under conditions sufficient for taking an antibody/antigen binding reaction; and

b) detecting a presence of the immune complex, which indicates a presence of the NT-proBNP in the test sample.

In this embodiment, the binding protein may be labeled with an indicator that shows signal intensity, so that the complex can be easily detected.

In one or more embodiments, in step a), the immune complex further includes a second antibody that binds to the binding protein;

In one or more embodiments, in step a), the immune complex further includes a second antibody that binds to the NT-proBNP:

in this embodiment, the binding protein forms a paired antibody with the second antibody in the form of a first antibody for binding to different epitopes of NT-proBNP;

the second antibody may be labeled with an indicator that shows signal intensity, so that the complex can be easily detected.

In one or more embodiments, in step a), the immune complex further includes a second antibody that binds to the NT-proBNP;

In this embodiment, the binding protein serves as an antigen of the second antibody, and the second antibody can be labeled with an indicator that shows signal intensity, so that the complex can be easily detected.

In one or more embodiments, the indicator for displaying signal intensity includes any one of fluorescent substance, quantum dot, digoxigenin-labeled probe, biotin, radioisotope, radiocontrast agent, paramagnetic ion fluorescent microsphere, electron dense substance, chemiluminescent marker, ultrasound contrast agent, photosensitizer, colloidal gold or enzyme.

In one or more embodiments, the fluorescent substance includes any one of Alexa 350, Alexa 405, Alexa 430, Alexa 488, Alexa 555, Alexa 647, AMCA, aminoacridine, BODIPY 630/650, BODIPY 650/665, BODIPY-FL, BODIPY-R6G, BODIPY-TMR, BODIPY-TRX, 5-carboxy-4′,5′-dichloro-2′,7′-dimethoxyfluorescein, 5-carboxy-2′,4′,5′,7′-tetrachlorofluorescein, 5-carboxyfluorescein, 5-carboxyrhodamine, 6-carboxyrhodamine, 6-carboxytetramethylrhodamine, Cascade Blue, Cy2, Cy3, Cy5, Cy7, 6-FAM, dansyl chloride, fluorescein, HEX, 6-JOE, NBD (7-nitrobenzo-2-oxa-1,3-diazole), Oregon Green 488, Oregon Green 500, Oregon Green514, Pacific Blue, phthalic acid, terephthalic acid, isophthalic acid, cresol solid violet, cresol blue violet, brilliant cresol blue, p-aminobenzoic acid, erythrosine, phthalocyanine, azomethine, cyanine, xanthine, succinylfluorescein, rare earth metal cryptate, tris-bispyridyldiamine europium, europium cryptate or chelate, diamine, biscyanin, La Jolla blue dye, allophycocyanin, alococyanin B, phycocyanin C, phycocyanin R, thiamine, phycoerythrin, phycoerythrin R, REG, rhodamine green, rhodamine isothiocyanate, rhodamine red, ROX, TAMRA, TET, TRIT (tetramethylrhodamine isothiol), tetramethylrhodamine and Texas Red.

In one or more embodiments, the radioisotope includes any one of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ⁹⁴mTc, ⁹⁴Tc, ⁹⁹mTc, ¹²⁰I, ¹²³I, ¹²⁵I, ¹³¹I, ¹⁵⁴⁻¹⁵⁸Gd, ³²P, ¹¹C ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb and ⁸³Sr.

In one or more embodiments, the enzyme includes any one of horseradish peroxidase, alkaline phosphatase, and glucose oxidase.

In one or more embodiments, the fluorescent microsphere is a polystyrene fluorescent microsphere, and the rare earth fluorescent ion europium is wrapped inside.

According to an aspect of the present disclosure, the present disclosure further relates to a kit, including the above binding protein.

The present disclosure further relates to use of the binding protein described herein in diagnosing a disease related to NT-proBNP.

As used herein, the term “a disease related to NT-proBNP” refers to a disease in which NT-proBNP, including its protein or encoding nucleic acid, is used as a marker. In particular, in one or more embodiments of the present disclosure, a disease related to NT-proBNP may refer to a disease characterized by an increase in the level of NT-proBNP in blood, such as whole blood, plasma or serum.

The present disclosure further relates to a method for diagnosing a disease related to NT-proBNP or evaluating cardiac function, the method including:
A) contacting a sample from a subject with the binding protein described in the present disclosure for a binding reaction under conditions sufficient for taking a binding reaction; and

B) detecting the immune complex produced by the binding reaction,

wherein the presence of the immune complex indicates the presence of a disease related to NT-proBNP or indicates the level of the cardiac function.

In one or more embodiments, the method is based on fluorescence immunoassay, chemiluminescence, colloidal gold immunoassay, radioimmunoassay, and/or enzyme-linked immunoassay.

In one or more embodiments, the sample is selected from at least one of whole blood, peripheral blood, serum, plasma or myocardial tissue.

In one or more embodiments, the subject is mammal, preferably primate, more preferably human.

In one or more embodiments, the disease related to NT-proBNP is a cardiac disease.

In one or more embodiments, the disease related to NT-proBNP is selected from heart failure, cardiac insufficiency, cardiogenic dyspnea, pulmonary dyspnea, acute coronary syndrome, or a combination thereof.

In one or more embodiments, the heart failure is cardiogenic heart failure or non-cardiac heart failure.

The embodiments of the present disclosure will be described in detail below together with examples, but those skilled in the art will understand that the following examples are only used to illustrate the present disclosure, and should not be regarded as limiting the scope of the present disclosure. If the specific condition is not indicated in the examples, it shall be carried out in accordance with the conventional condition or the condition recommended by the manufacturer. The reagents or instruments used without the manufacturer are al conventional products that can be purchased commercially.

### Example 1

This example provides an exemplary preparation method for a recombinant antibody of anti-human NT-proBNP.

S1. Construction of an Expression Plasmid:

In this example, the restriction endonuclease and Prime Star DNA polymerase were purchased from Takara Company;

MagExtractor-RNA extraction kit was purchased from TOYOBO Company;

BD SMART™ RACE cDNA Amplification Kit was purchased from Takara Company;

pMD-18T vector was purchased from Takara Company;

the plasmid extraction kit was purchased from Tiangen Company;

The primer synthesis and gene sequencing were completed by Invitrogen Company;

the hybridoma cell strain that secreted the Anti-NT-proBNP 889 monoclonal antibody was an existing hybridoma cell strain and was resuscitated for use.

S11, Design and Synthesis of Primers:

Amplification of 5′RACE forward primers for heavy chains and light chains:

SMARTER II A oligonucleotide:
(SEQ ID NO: 19)
5′>AAGCAGTGGTATCAACGCAGAGTACXXXXX<3′;
5′-RACE CDS primer (5′-CDS):
(SEQ ID NO: 20)
5′>(T)₂₅VN<3′(N = A, C, G, or T; V = A, G, or C);
Universal Primer A mixture (UPM):
(SEQ ID NO: 21)
5′>CTAATACGACTCACTATAGGGCAAGCAGTGGTATCAACGCAGAG
T<3′;
Nested universal primer A (NUP):
(SEQ ID NO: 22)
5′>AAGCAGTGGTATCAACGCAGAGT<3′;
mkR:
(SEQ ID NO: 23)
5′>CCCGAATTCCTAAGAACACTCTGAGGGCTTCACTG<3′;
mHR:
(SEQ ID NO: 24)
5′>CCCGAATTCTCATTTACCCGGAGGCTTAGAGAT<3′.

S12, Gene Cloning and Sequencing for Antibody Variable Region:

The RNA was extracted from the hybridoma cell strain secreting Anti-NT-proBNP 8B9 monoclonal antibody, and the first chain cDNA was synthesized using the SMARTER™ RACE cDNA Amplification Kit and the SMARTER II A oligonucleotide and 5′-CDS primers in the kit. The obtained first chain cDNA product was used as a template for PCR amplification. Light chain genes were amplified with universal primer A mix (UPM), nested universal primer A (NUP) and mkR, and heavy chain genes were amplified with universal primer A mix (UPM), nested universal primer A (NUP) and mHR. Wherein, the primer pair for the light chain amplified the target band of about 0.7 KB, and the primer pair for the heavy chain amplified the target band of about 1.4 KB. The product purified and recovered by agarose gel electrophoresis was subjected to A-addition reaction with rTaq DNA polymerase, followed by inserting into pMD-18T vector and transforming Into DH5a competent cells. After the growth of the colony, 4 clones of the heavy chain and light chain gene clones were picked and sent to Invitrogen Company for sequencing.

S13, Sequence Analysis of Variable Region Genes of Anti-NT-proBNP 889 Antibody:

The gene sequence obtained by the above sequencing was analyzed in the IMGT antibody database, and the amplified genes were analyzed to confirm that the heavy chain and light chain primers were correct, and the light chain amplified genes by using VNTI11.5 software. Among the gene fragments amplified by the light chain, the VL gene sequence was 420 bp, belonging to the VkII gene family, with a 60 bp leader peptide sequence in front of it; among the gene fragments amplified by the heavy chain primer pair, the VH gene sequence was 402 bp, belonging to the VH1 gene family, with a 57 bp leader peptide sequence in front of it.

S14, Construction of Recombinant Antibody Expression Plasmid:

pcDNAT™ 3.4 TOPO@ vector was a constructed recombinant antibody eukaryotic expression vector. The expression vector had been introduced with HindIII, BamHI, EcoRI and other polyclonal restriction sites, named as pcDNA3.4A expression vector, and then referred to as 3.4A expression vector; according to the above sequencing results of antibody variable region genes in pMD-18T, the VL and VH gene-specific primers of Anti-NT-proBNP 8B9 antibody were designed, with HindIII and EcoRI restriction sites and protective bases at both terminals, with the primers as follows:

NT-proBNP 8B9-HF:

(SEQ ID NO: 25)

5′>CCCAAGCTTGCCACCATGAACCCACTGTGGACCCTCCTCTTT<3;
NT-proBNP 8B9-HR:

(SEQ ID NO: 26)

5′>CCCGAATTCTCATTTACCCGGAGGCTTAGAGAT<3′;
NT-proBNP 8B9-LF:

(SEQ ID NO: 27)

5′>CCCAAGCTTGCCACCATGGCCTGGTCCCCTCTGCTCCTCAC<3′;
NT-proBNP 8B9-LR:

(SEQ ID NO: 28)

5′>CCCGAATTCCTAAGAACACTCTGAGGGCTTCACTG<3′;

The 0.7 KB light chain gene fragment and 1.4 kb heavy chain gene fragment were amplified by PCR amplification method. The heavy chain and light chain gene fragments were digested with HindIII/EcoRI, respectively, and the 3.4A vector was digested with HindIII/EcoRI. After the fragment and vector being purified and recovered, the heavy chain gene and light chain gene were linked to the 3.4A expression vector, respectively, to obtain recombinant expression plasmids of heavy chain and light chain.

S2. Screening of Stable Cell Strains

S21 Recombinant Antibody Expression Plasmid was Transiently Transfected into CHO Cells to Confirm the Activity

Plasmids were diluted with ultrapure water to 400 ng/ml, and the CHO cells were adjusted to 1.43×107 cells/ml in a centrifuge tube. 100 μl of plasmids were mixed with 700 μl of cells, which were transferred to the electrotransfection cuvette for electrotransfection. The samples were taken and counted on Day 3, 5 and 7, and were collected on Day 7 for testing.

Recombinant NT antigen (self-produced, 161213) was diluted with coating solution to the specified concentration at 100 μL per well overnight at 4° C.; which was washed twice with washing solution and patted dry on the next day; with addition of blocking solution (20% BSA+80% PBS) at 120 μL per well at 37° C. for 1 h and patted dry; added the diluted cell supernatant, 100 μL/well at 37° C. for 30 min (partial supernatant for 1 h); washed 5 times with washing solution and patted dry; added rabbit anti-goat IgG-HRP 100 μL per well at 37 C for 30 min; washed 5 times with washing solution and patted dry; added color developing solution A (50 μL/well), added color developing solution B (50 μL/well) for 10 min; added stop solution, 50 μL/well; followed by reading the OD value at 450 nm (reference 630 nm) on the microplate reader. The results showed that the OD of the reaction was still greater than 1.0 after the cell supernatant being diluted 1000 times, and the reaction OD of the wells without the cell supernatant was less than 0.1, indicating that the antibody produced after transient transfection with the plasmid was active against recombinant NT protein.

S22 Linearization of Recombinant Antibody Expression Plasmid

Preparation of the following reagents: Buffer 50 μl, DNA 100 μg/tube, Puv I enzyme 10 μl, supplemented with sterile water to 500 μl, for digestion overnight at 37° C. in the water bath; which was extracted with an equal volume of phenol/chloroform/isoamyl alcohol (lower layer) 25:24:1 and then chloroform (aqueous phase) in sequence; precipitated on ice with 0.1 times volume (aqueous phase) 3M sodium acetate and 2 times volume of ethanol, and the obtained precipitate was rinsed with 70% ethanol to remove the organic solvent, followed by thawing with an appropriate amount of sterilized water until the ethanol evaporated completely to be determined the concentration finally.

S23 Stable Transfection of Recombinant Antibody Expression Plasmid and Selection of Stable Cell Strains Under Pressure

Plasmids were diluted with ultrapure water to 400 ng/ml, and the CHO cells were adjusted to 1.43×10⁷ cells/ml in a centrifuge tube. 100 μl of plasmids were mixed with 700 μl of cells, which were transferred to the electrotransfection cuvette for electrotransfection and counted on the next day, followed by culturing at 25 μmol/L MSX 96 wells under pressure for about 25 days.

The clonal wells with labeled cells were observed under a microscope, and recorded the confluence; from which the culture supernatant were taken and sent for testing; and cell strains with high antibody concentration and relative concentration were transferred to 24 wells, and then transferred to 6 wells in about 3 days; followed by preservation and batch culture with adjustment the cell density to 0.5×10⁶ cells/ml, from which 2.2 ml for batch culture at the cell density of 0.3×10⁶ cells/ml, and 2 ml for preservation; and the supernatant from 6 wells batch culture for 7 days was sent for testing, from which the cell strain with smaller antibody concentration and cell diameter was selected for TPP preservation and passage.

S3. Production of Recombinant Antibody

S31 Cell Expansion Culture

After resuscitation, the cells were cultured in a 125 ml shake flask with a 30 ml inoculation volume and 100% Dynamis medium, placed in a shaker with a rotation speed of 120 r/min at 37° C., and a carbon dioxide of 8%. Cells were cultured for 72 h and inoculated at an inoculation density of 500,000 cells/ml for expansion culture, in which the expansion volume was calculated according to production requirements, using 100% Dynamis medium. Then the culture was expanded every 72 h. When the cell mass met the production requirements, the inoculation density was strictly controlled to about 500,000 cells/ml for production.

S32 Production in the Shake Flask and Purification

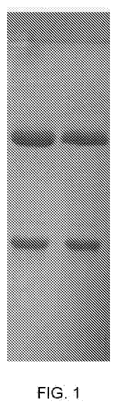

Shake flask parameters: rotation at a speed of 120 r/min, at a temperature of 37° C., and with carbon dioxide of 8%. Feeding: feeding every day was started at after it is cultured for 72 h in the shake flask. HyClone™ Cell Boost™ Feed 7a was fed 3% of the initial culture volume every day, and Feed 7b with fed every thousandth of the initial culture volume, until to Day 12 (feeding on Day 12). Glucose was supplemented at 3 g/L on Day 6. Samples were collected on Day 13. Then the affinity purification was carried out by a proteinA affinity chromatography column. 4 μg of purified antibodies was taken for reducibility SDS-PAGE, and 4 μg of foreign control antibodies was used as a control. The electrophoretogram was shown in FIG. 1. After reducibility SDS-PAGE, two bands were displayed, one Mr was 50 KD (heavy chain), and the other Mr was 28 KD (light chain).

### Example 2

Antibody Affinity Analysis and Activity Identification

The antibody obtained in Example 1 was analyzed to have a light chain as shown in SEQ ID NO: 11 and a heavy chain as shown in SEQ ID NO: 12.

After analysis, the complementarity determining region (WT) of the heavy chain:

CDR-VH1 was G-P(X1)-S-I(X2)-T-T-Y-Y-I(X3)-D;

CDR-VH2 was M-T-K-D-A(X1)-N-A-V-H-Q(X2)-P-T-I(X3)-R-S;

CDR-VH3 was V-K(X1)-G-I(X2)-I-D-W(X3)-G;

the complementarity determining region of the light chain:

CDR-VL1 was G-S-S-D-Q(X1)-V-G-P(X2)-G-D-Y-V(X3)-N;

CDR-VL2 was I-F-A(X1)-A-T(X2)-S-R-I(X3)-R-G;

CDR-VL3 was G-S-P(X1)-N-S-R-GG(X2)-Y-V-W(X3)-G;

wherein, X1, X2, and X3 were all mutation sites. Table 1 Mutation sites related to antibody activity

| | CDR-VH1 | CDR-VH2 | CDR-VH3 | CDR-VL1 | CDR-VL2 | CDR-VL3 |
|---|---|---|---|---|---|---|
| Site | X1 | X1 | X1 | X2 | X1 | X3 |
| WT | P | A | K | P | A | w |
| Mutation 1 | F | G | R | F | G | F |
| Mutation 2 | P | G | R | P | G | F |
| Mutation 3 | F | A | R | F | A | F |
| Mutation 4 | P | G | K | F | G | w |

The CDR sites in WT was mutated by the inventor as described above to obtain antibodies with better activity.

Recombinant NT antigen was diluted with coating solution to 1 μg/ml a microplate overnight at 4° C.; which was washed twice with washing solution and patted dry on the next day; with addition of blocking solution (20% BSA+80% PBS) at 120 μL per well at 37° C. for 1 h and patted dry; added the diluted NT monoclonal antibody, 100 μL/well at 37° C. for 30 min (partial supernatant for 1 h); washed 5 times with washing solution and patted dry; added rabbit anti-goat IgG-HRP 100 NL per well at 37° C. for 30 min; washed 5 times with washing solution and patted dry; added color developing solution A (50 μL/well), added color developing solution B (50 μL/well) for 10 min; added stop solution, 50 μL/well; followed by reading the OD value at 450 nm (reference 630 nm) on the microplate reader.

TABLE 2
Antibody activity analysis data

| Sample | | | | | |
|---|---|---|---|---|---|
| concentration | | Muta- | Muta- | Muta- | Muta- |
| ng/ml | WT | tion 1 | tion 2 | tion 3 | tion 4 |
| 111.11 | 1.528 | 1.827 | 1.772 | 1.764 | 1.724 |
| 37.04 | 1.236 | 1.568 | 1.526 | 1.533 | 1.501 |
| 12.35 | 0.732 | 1.110 | 1.031 | 1.022 | 0.959 |
| 4.12 | 0.312 | 0.564 | 0.523 | 0.520 | 0.489 |
| 1.37 | 0.218 | 0.355 | 0.353 | 0.347 | 0.300 |
| 0 | 0.067 | 0.121 | 0.089 | 0.076 | 0.115 |

From above tables, mutation 1 was used as the framework sequence to screen for mutation sites with better potency due to having best activity (ensure that the antibody activity obtained by screening was similar to that of mutation 1, having the antibody activity of ±10%), some of the results were as follows.

TABLE 3
Mutation sites related to antibody affinity

| | CDR-VH1 | CDR-VH2 | CDR-VH3 | CDR-VL1 | CDR-VL2 | CDR-VL3 |
|---|---|---|---|---|---|---|
| Site | X2/X3 | X2/X3 | X2/X3 | X1/X3 | X2/X3 | X1/X2 |
| Mutation 1 | I/I | Q/I | I/W | Q/V | T/I | P/GG |
| Mutation 1-1 | I/L | N/I | I/F | Q/L | T/L | P/N |
| Mutation 1-2 | I/V | Q/L | L/W | Q/I | T/V | A/GG |
| Mutation 1-3 | L/I | N/L | L/F | N/V | Y/I | A/N |
| Mutation 1-4 | L/L | Q/V | V/W | N/L | Y/L | G/GG |
| Mutation 1-5 | L/V | N/V | V/F | N/I | Y/V | G/N |
| Mutation 1-6 | V/I | N/V | I/W | N/I | S/I | P/N |
| Mutation 1-7 | V/L | Q/V | I/F | N/L | S/L | G/N |
| Mutation 1-8 | V/V | N/L | L/W | N/V | S/V | G/GG |
| Mutation 1-9 | I/I | Q/L | L/F | Q/I | S/I | A/GG |
| Mutation 1-10 | I/L | N/I | V/W | Q/L | S/L | A/N |
| Mutation 1-11 | I/V | Q/I | V/F | Q/V | S/V | P/GG |
| Mutation 1-12 | L/I | Q/I | I/W | Q/V | Y/I | P/GG |
| Mutation 1-13 | L/L | N/I | I/F | Q/L | Y/L | P/N |
| Mutation 1-14 | L/V | Q/L | L/W | Q/I | Y/V | A/GG |
| Mutation 1-15 | V/I | N/L | L/F | N/V | T/I | A/N |
| Mutation 1-16 | V/L | Q/V | V/W | N/L | T/L | G/GG |
| Mutation 1-17 | V/V | N/V | V/F | N/I | T/V | G/N |
| Mutation 1-18 | I/I | N/V | I/W | N/I | T/I | P/N |
| Mutation 1-19 | I/L | Q/V | I/F | N/L | T/L | G/N |
| Mutation 1-20 | I/V | N/L | L/W | N/V | T/V | G/GG |
| Mutation 1-21 | L/I | Q/L | L/F | Q/I | Y/I | A/GG |
| Mutation 1-22 | L/L | N/I | V/W | Q/L | Y/L | A/N |
| Mutation 1-23 | L/V | Q/I | V/F | Q/V | Y/V | P/GG |
| Mutation 1-24 | V/I | Q/I | I/W | Q/V | S/I | P/GG |
| Mutation 1-25 | V/L | N/I | I/F | Q/L | S/L | P/N |
| Mutation 1-26 | V/V | Q/L | L/W | Q/V | S/V | A/GG |
| Mutation 1-27 | I/I | N/L | L/F | N/V | S/I | A/N |
| Mutation 1-28 | I/L | Q/V | V/W | N/L | S/L | G/GG |
| Mutation 1-29 | I/V | N/V | V/F | N/I | S/V | G/N |
| Mutation 1-30 | L/I | N/V | I/W | N/I | T/I | P/N |
| Mutation 1-31 | L/L | Q/V | I/F | N/L | T/L | G/N |
| Mutation 1-32 | L/V | N/L | L/W | N/V | T/V | G/GG |
| Mutation 1-33 | V/I | Q/L | L/F | Q/I | Y/I | A/GG |
| Mutation 1-34 | V/L | N/I | V/W | Q/L | Y/L | A/N |
| Mutation 1-35 | V/V | Q/I | V/F | Q/V | Y/V | P/GG |
| Mutation 1-36 | I/I | Q/I | I/W | Q/V | T/I | P/GG |
| Mutation 1-37 | I/L | N/I | I/F | Q/L | T/L | A/N |
| Mutation 1-38 | I/V | Q/L | L/W | Q/I | T/V | G/GG |
| Mutation 1-39 | L/I | N/L | L/F | N/V | Y/I | G/N |
| Mutation 1-40 | L/L | Q/V | V/W | N/L | Y/L | P/N |
| Mutation 1-41 | V/I | N/V | I/W | N/I | Y/V | G/N |
| Mutation 1-42 | V/I | N/V | I/W | N/I | S/I | G/GG |
| Mutation 1-43 | V/L | Q/V | I/F | N/L | S/L | A/GG |
| Mutation 1-44 | V/V | N/L | L/W | N/V | S/V | A/N |
| Mutation 1-45 | I/I | Q/L | L/F | Q/I | S/I | P/GG |
| Mutation 1-46 | I/L | N/I | V/W | Q/L | S/L | P/GG |
| Mutation 1-47 | I/V | Q/I | V/F | Q/V | S/V | P/N |
| Mutation 1-48 | L/I | Q/I | I/W | Q/V | T/I | P/GG |
| Mutation 1-49 | L/L | N/I | I/F | Q/L | T/L | P/N |
| Mutation 1-50 | L/V | Q/L | L/W | Q/I | T/V | A/GG |
| Mutation 1-51 | V/I | N/L | L/F | N/V | Y/I | A/N |
| Mutation 1-52 | V/L | Q/V | V/W | N/L | Y/L | G/GG |
| Mutation 1-53 | V/V | N/V | V/F | N/I | Y/V | G/N |

Affinity Analysis

Data from the enzyme immunoassay indirect method were analyzed in the same way as the activity identification, with four gradients of 3 μg/ml, 1.5 μg/ml, 0.75 μg/ml and 0.375 μg/ml for coating; the antibody was diluted from 1000 ng/ml by 2 times to 0.977 ng/ml for loading sample. OD values corresponding to different antibody concentrations at different coating concentrations were obtained. Under the same coating concentration, data were plotted logarithmically with the antibody concentration as the abscissa and the OD value as the ordinate. According to the fitting equation, the antibody concentration at 50% of the maximum OD value was calculated, which was used Into the formula: K=(n−1)/(2×(n×Ab′−Ab)) to calculate the reciprocal of the affinity constant, wherein Ab and Ab′ represented the antibody concentration at 50% of the maximum OD value at the corresponding coating concentration (Ag, Ag′), n=Ag/Ag′; every two coating concentrations could be combined to calculate a K value, and finally six K values could be obtained, which were averaged followed by being calculated their reciprocals to obtain the affinity constant KID.

TABLE 4
Affinity analysis data

| No. | K_{D} (M) |
|---|---|
| Mutation 1 | 1.73E−09 |
| Mutation 1-1 | 2.51E−09 |
| Mutation 1-2 | 2.61E−09 |
| Mutation 1-3 | 1.52E−09 |
| Mutation 1-4 | 2.07E−09 |
| Mutation 1-5 | 6.10E−09 |
| Mutation 1-6 | 1.17E−09 |
| Mutation 1-7 | 1.53E−09 |
| Mutation 1-8 | 2.22E−09 |
| Mutation 1-9 | 1.56E−09 |
| Mutation 1-10 | 3.20E−09 |
| Mutation 1-11 | 1.52E−09 |
| Mutation 1-12 | 2.75E−09 |
| Mutation 1-13 | 2.09E−09 |
| Mutation 1-14 | 8.75E−10 |
| Mutation 1-15 | 1.76E−09 |
| Mutation 1-16 | 3.27E−09 |
| Mutation 1-17 | 3.39E−09 |
| Mutation 1-18 | 1 73E−09 |
| Mutation 1-19 | 1.51E−09 |
| Mutation 1-20 | 2.61E−09 |
| Mutation 1-21 | 2.53E−09 |
| Mutation 1-22 | 2.07E−09 |
| Mutation 1-23 | 8.10E−10 |
| Mutation 1-24 | 1.17E−09 |
| Mutation 1-25 | 2.16E−09 |
| Mutation 1-26 | 1.51E−09 |
| Mutation 1-27 | 8.55E−10 |
| Mutation 1-28 | 2.25E−09 |
| Mutation 1-29 | 1.49E−09 |
| Mutation 1-30 | 2.66E−09 |
| Mutation 1-31 | 1.84E−09 |
| Mutation 1-32 | 1 52E−09 |
| Mutation 1-33 | 2.60E−09 |
| Mutation 1-34 | 3.12E−09 |
| Mutation 1-35 | 1.51E−09 |
| Mutation 1-36 | 5.64E−09 |
| Mutation 1-37 | 1.57E−09 |
| Mutation 1-38 | 1.82E−09 |
| Mutation 1-39 | 2.45E−09 |
| Mutation 1-40 | 3.47E−09 |
| Mutation 1-41 | 2.82E−09 |
| Mutation 1-42 | 2.22E−09 |
| Mutation 1-43 | 2.93E−09 |
| Mutation 1-44 | 1.26E−09 |
| Mutation 1-45 | 1.53E−09 |
| Mutation 1-46 | 3.49E−09 |
| Mutation 1-47 | 2.79E−09 |
| Mutation 1-48 | 2.02E−09 |
| Mutation 1-49 | 6.83E−09 |
| Mutation 1-50 | 2.16E−09 |
| Mutation 1-51 | 9.44E−10 |
| Mutation 1-52 | 2.71E−09 |
| Mutation 1-53 | 2.06E−09 |

From Table 4, the mutation sites listed in Table 3 had little effect on the affinity of the antibody.

In order to verify the above results, the above experiment was repeated with WT as the framework sequence to verify the affinity of the mutation site, with some of the results as follows.

TABLE 5
Mutations with WT as the framework

| | CDR-VH1 | CDR-VH2 | CDR-VH3 | CDR-VL1 | CDR-VL2 | CDR-VL3 |
|---|---|---|---|---|---|---|
| Site | X2/X3 | X2/X3 | X2/X3 | X1/X3 | X2/X3 | X1/X2 |
| WT | I/I | Q/I | I/F | Q/V | T/I | P/GG |
| WT 1-1 | V/I | N/I | V/W | N/I | Y/I | A/N |
| WT 1-2 | L/I | Q/L | V/W | N/L | S/I | P/N |
| WT 1-3 | I/L | Q/I | L/F | N/V | T/I | A/N |
| WT 1-4 | V/V | Q/L | V/F | Q/V | T/I | G/GG |
| WT 1-5 | L/L | N/V | L/F | Q/L | S/I | A/N |
| WT 1-6 | I/V | Q/I | L/W | Q/L | S/L | A/GG |
| WT 1-7 | V/L | Q/V | V/W | N/I | Y/L | G/GG |
| WT 1-8 | L/V | Q/V | I/W | N/I | Y/V | P/N |
| WT 1-9 | V/I | Q/L | L/W | N/V | Y/L | G/GG |

TABLE 6
Affinity analysis data

| No. | KD (M) |
|---|---|
| WT | 3.02E−09 |
| WT 1-1 | 1.78E−08 |
| WT 1-2 | 3.48E−09 |
| WT 1-3 | 2.53E−09 |
| WT 1-4 | 3.02E−09 |
| WT 1-5 | 2.26E−08 |
| WT 1-6 | 6.14E−09 |
| WT 1-7 | 5.05E−09 |
| WT 1-8 | 4.78E−09 |
| WT 1-9 | 2.47E−09 |

According to the analysis from Table 5 and Table 6, the above mutation site had little correlation with other sites, provided that the activity of the antibody was ensured.

### Example 3 Stability Analysis

The antibody was placed at 4 C (fridge), −80° C. (fridge), 37 C (incubator) for 21 days, with taking samples at Day 7, Day 14 and Day 21 for state observation, and the activities of samples at Day 7 were detected. The results showed that under the three assessment conditions, there was no significant protein status change after the antibody was placed for 21 days, and the activity did not decrease with the increasement of the assessment temperature, indicating that the self-produced antibody was stable. The following table showed the OD results of enzyme immunoassay test for 21 days for mutation 1.

TABLE 7
Stability analysis data

| | Sample concentration (ng/ml) | | |
|---|---|---|---|
| | 111.11 | 12.35 | 0 |
| 4° C., sample | 1.726 | 0.864 | 0.047 |
| on Day 21 | | | |
| −80° C., sample | 1.845 | 0.976 | 0.036 |
| on Day 21 | | | |
| 37° C., sample | 1.813 | 0.843 | 0.033 |
| on Day 21 | | | |

Furthermore, 8 randomly selected mutant antibodies were tested for stability; the above antibodies were stored at 37° C. for 72 hours, and after removal, they were tested with the same negative and positive quality control samples under the same test conditions with the same batch of antibodies stored at 4° C. for 72 hours. The test method was the same as the antibody activity analysis method used in the above examples. The linearity of each group of antibodies could reach more than 99.50%, and the CV value was less than 10%, which showed that the above antibodies had excellent stability.

### Example 4 Evaluation for Pairing Performance

The above antibodies in Table 4 with another internal antibody (antibody paired with the original WT sequence antibody) were verified using a paired antibody experiment by the applicant. Their specificities maintained at the original high level verified by the double antibody sandwich method paired experiment, but due to the increased activity and affinity of the mutant antibody, it exhibited higher sensitivity.

The mutant 1 antibody and WT antibody were used as coating antibodies, accompanied with another strain as a labeled NT-oroBNP antibody. The performance difference was compared on the chemiluminescence evaluation platform, with the specific performance shown in the following table:

TABLE 8
Evaluation table for pairing performance

| Performance | Decection | | | | Detection |
|---|---|---|---|---|---|
| index | range | Sensitivity | | Linear | rate |
| Mutation 1 | 0-50000 pg/ml | 5.8 | pg/ml | 0.97689 | 100% |
| WT | 0-50000 pg/ml | 6 | pg/ml | 0.95638 | 99% |

Finally, it should be noted that the above embodiments are only used to illustrate the technical solutions of the present disclosure, not to limit them; although the present disclosure has been described in detail with reference to the foregoing embodiments, but those of ordinary skill in the art should understand that: the technical solutions recorded in the foregoing embodiments can still be modified, or some or all of the technical features can be equivalently replaced; and these modifications or replacements do not cause the essence of the corresponding technical solutions to deviate from the scope of the technical solutions of the embodiments in the present disclosure.

### INDUSTRIAL APPLICABILITY

The binding protein described in the present disclosure has strong activity and high affinity to human NT-proBNP. The binding protein described in the present disclosure can be used to diagnose a disease related to NT-proBNP, such as heart failure, and can be used to evaluate cardiac function with high sensitivity and specificity.

What is claimed is:

1. An isolated binding protein comprising an antigen-binding domain, wherein the antigen-binding domain comprises at least one complementarity determining region (CDR) selected from the following amino acid sequences: or has at least 80% sequence identity with the complementarity determining region of the following amino acid sequences and has an affinity of K_{D}≤2.26×10⁻⁸ to a N-terminal pro-brain natriuretic peptide (NT-proBNP);
the complementarity determining region CDR-VH1 is G-X1-S-X2-T-T-Y-Y-X3-D, wherein
X1 is F or P, X2 is I, V or L, and X3 is I, V or L;
the complementarity determining region CDR-VH2 is M-T-K-D-X1-N-A-V-H-X2-P-T-X3-R-S, wherein
X1 is G or A, X2 is Q or N, and X3 is I, V or L;
the complementarity determining region CDR-VH3 is V-X1-G-X2-1-D-X3-G, wherein
X1 is R or K, X2 is I, V or L, and X3 is F or W;
the complementarity determining region CDR-VL1 is G-S-S-D-X1-V-G-X2-G-D-Y-X3-N, wherein
X1 is Q or N, X2 is F or P, and X3 is I, V or L;
the complementarity determining region CDR-VL2 is I-F-X1-A-X2-S-R-X3-R-G, wherein
X1 is G or A, X2 is T, Y or S, and X3 is 1, V or L;
the complementarity determining region CDR-VL3 is G-S-X1-N-S-R-X2-Y-V-X3-G, wherein
X1 is P, A or G, X2 is GG or N, and X3 is F or W;
preferably:
X1 is F in the complementarity determining region CDR-VH1;
X1 Is G in the complementarity determining region CDR-VH2;
X1 is R in the complementarity determining region CDR-VH3;
X2 is F in the complementarity determining region CDR-VL1;
X1 is G in the complementarity determining region CDR-VL2;
X3 is F in the complementarity determining region CDR-VL3;
preferably, X2 is I in the complementarity determining region CDR-VH1;
preferably, X2 is V in the complementarity determining region CDR-VH1;
preferably, X2 is L in the complementarity determining region CDR-VH1;
preferably, X3 is I in the complementarity determining region CDR-VH1;
preferably, X3 is V in the complementarity determining region CDR-VH1;
preferably, X3 is L in the complementarity determining region CDR-VH1;
preferably, X2 is Q in the complementarity determining region CDR-VH2;
preferably, X2 is N in the complementarity determining region CDR-VH2;
preferably, X3 is I in the complementarity determining region CDR-VH2;
preferably, X3 is V in the complementarity determining region CDR-VH2;
preferably, X3 is L in the complementarity determining region CDR-VH2;
preferably, X2 is I in the complementarity determining region CDR-VH3;
preferably, X2 is V in the complementarity determining region CDR-VH3;
preferably, X2 is L in the complementarity determining region CDR-VH3;
preferably, X3 is F in the complementarity determining region CDR-VH3;
preferably, X3 is W in the complementarity determining region CDR-VH3;
preferably, X1 is Q in the complementarity determining region CDR-VL1;
preferably, X1 is N in the complementarity determining region CDR-VL1;
preferably, X3 is I in the complementarity determining region CDR-VL1;
preferably, X3 is V in the complementarity determining region CDR-V 1;
preferably, X3 is L in the complementarity determining region CDR-V 1;
preferably, X2 is T in the complementarity determining region CDR-VL2;
preferably, X2 is Y in the complementarity determining region CDR-VL2;
preferably, X2 is S in the complementarity determining region CDR-VL2;
preferably, X3 is I in the complementarity determining region CDR-VL2;
preferably, X3 is V in the complementarity determining region CDR-VL2;
preferably, X3 is L in the complementarity determining region CDR-VL2;
preferably, X1 is P in the complementarity determining region CDR-VL3;
preferably, X1 is A in the complementarity determining region CDR-VL3;
preferably, X1 is G in the complementarity determining region CDR-VL3;
preferably, X2 is GG in the complementarity determining region CDR-VL3;
preferably, X2 is N in the complementarity determining region CDR-VL3.

2. The isolated binding protein comprising an antigen-binding domain according to claim 1, wherein the binding protein comprises at least 3 CDRs; alternatively, the binding protein comprises at least 6 CDRs.

3. The isolated binding protein comprising an antigen-binding domain according to claim 1, wherein the binding protein further comprises a constant region sequence of antibody;
preferably, the constant region sequence is a sequence of any one constant region selected from IgG1, IgG2, IgG3, IgG4, IgA, IgM, IgE, and IgD;
preferably, the constant region is derived from species consisted of cattle, horse, dairy cow, pig, sheep, goat, rat, mouse, dog, cat, rabbit, camel, donkey, deer, mink, chicken, duck, goose, turkey, gamecock or human;
preferably, the constant region is derived from the sheep;
a light chain constant region sequence is shown in SEQ ID NO: 9;
a heavy chain constant region sequence is shown in SEQ ID NO: 10.

4. An isolated nucleic acid molecule, the nucleic acid molecule being DNA or RNA, which encodes the binding protein according to claim 1.

5. A vector, comprising the nucleic acid according to claim 4.

6. A host cell transformed with the vector according to claim 5.

7. A method for producing the binding protein according to claim 1, the method comprising the steps of:
culturing a host cell comprising a nucleic acid encoding the binding protein in a culture medium and under suitable culture conditions, recovering such produced binding protein from the culture medium or from the cultured host cell.

8. (canceled)

9. A method for detecting NT-proBNP in a test sample, the method comprising:
a) contacting the NT-proBNP antigen in the test sample with the binding protein according to claim 1 to form an immune complex under conditions sufficient for taking an antibody/antigen binding reaction; and
b) detecting a presence of the immune complex, which indicates a presence of the NT-proBNP In the test sample;
preferably, in step a), the immune complex further comprises a second antibody that binds to the binding protein;
preferably, in step a), the immune complex further comprises a second antibody that binds to the NT-proBNP.

10. A kit, comprising the binding protein as defined in claim 1.

11. (canceled)

12. The method according to claim 9, wherein
the presence of the immune complex indicates the presence of a disease related to NT-proBNP or indicates the level of the cardiac function.

13. The method according to claim 9, wherein the method is based on fluorescence immunoassay, chemiluminescence, colloidal gold immunoassay, radioimmunoassay, and/or enzyme-linked immunoassay.

14. The method according to claim 9, wherein the sample is selected from at least one of whole blood, peripheral blood, serum, plasma or myocardial tissue.

15. The method according to claim 12, wherein a subject is mammal, preferably primate, more preferably human.

16. The method according to claim 12, wherein the disease related to NT-proBNP is a cardiac disease.

17. The method according to claim 12, wherein the disease related to NT-proBNP is selected from heart failure, cardiac insufficiency, cardiogenic dyspnea, pulmonary dyspnea, acute coronary syndrome, or a combination thereof.

18. The method according to claim 17, wherein the heart failure is cardiogenic heart failure or non-cardiac heart failure.

19. The isolated binding protein comprising an antigen-binding domain according to claim 1, wherein a mutation site of each complementarity determining region is selected from any one of the following nutation combinations:

| | CDR-VH1 | CDR-VH2 | CDR-VH3 | CDR-VL1 | CDR-VL2 | CDR-VL3 |
|---|---|---|---|---|---|---|
| Site | X2/X3 | X2/X3 | X2/X3 | X1/X3 | X2/X3 | X1/X2 |
| Mutation | I/I | Q/I | I/W | Q/V | T/I | P/GG |
| combination 1 | | | | | | |
| Mutation | I/L | N/I | I/F | Q/L | T/L | P/N |
| combination 2 | | | | | | |
| Mutation | I/V | Q/L | L/W | Q/I | T/V | A/GG |
| combination 3 | | | | | | |
| Mutation | L/I | N/L | L/F | N/V | Y/I | A/N |
| combination 4 | | | | | | |
| Mutation | L/L | Q/V | V/W | N/L | Y/L | G/GG |
| combination 5 | | | | | | |
| Mutation | L/V | N/V | V/F | N/I | Y/V | G/N |
| combination 6 | | | | | | |
| Mutation | V/I | N/V | I/W | N/I | S/I | P/N |
| combination 7 | | | | | | |
| Mutation | V/L | Q/V | I/F | N/L | S/L | G/N |
| combination 8 | | | | | | |
| Mutation | V/V | N/L | L/W | N/V | S/V | G/GG |
| combination 9 | | | | | | |
| Mutation | I/I | Q/L | L/F | Q/I | S/I | A/GG |
| combination 10 | | | | | | |
| Mutation | I/L | N/I | V/W | Q/L | S/L | A/N |
| combination 11 | | | | | | |
| Mutation | I/V | Q/I | V/F | Q/V | S/V | P/GG |
| combination 12 | | | | | | |
| Mutation | L/I | Q/I | I/W | Q/V | Y/I | P/GG |
| combination 13 | | | | | | |
| Mutation | L/L | N/I | I/F | Q/L | Y/L | P/N |
| combination 14 | | | | | | |
| Mutation | L/V | Q/L | L/W | Q/I | Y/V | A/GG |
| combination 15 | | | | | | |
| Mutation | V/I | N/L | L/F | N/V | T/I | A/N |
| combination 16 | | | | | | |
| Mutation | V/L | Q/V | V/W | N/L | T/L | G/GG |
| combination 17 | | | | | | |
| Mutation | V/V | N/V | V/F | N/I | T/V | G/N |
| combination 18 | | | | | | |
| Mutation | I/I | N/V | I/W | N/I | T/I | P/N |
| combination 19 | | | | | | |
| Mutation | I/L | Q/V | I/F | N/L | T/L | G/N |
| combination 20 | | | | | | |
| Mutation | I/V | N/L | L/W | N/V | T/V | G/GG |
| combination 21 | | | | | | |
| Mutation | L/I | Q/L | L/F | Q/I | Y/I | A/GG |
| combination 22 | | | | | | |
| Mutation | L/L | N/I | V/W | Q/L | Y/L | A/N |
| combination 23 | | | | | | |
| Mutation | L/V | Q/I | V/F | Q/V | Y/V | P/GG |
| combination 24 | | | | | | |
| Mutation | V/I | Q/I | I/W | Q/V | S/I | P/GG |
| combination 25 | | | | | | |
| Mutation | V/L | N/I | I/F | Q/L | S/L | P/N |
| combination 26 | | | | | | |
| Mutation | V/V | Q/L | L/W | Q/V | S/V | A/GG |
| combination 27 | | | | | | |
| Mutation | I/I | N/L | L/F | N/V | S/I | A/N |
| combination 28 | | | | | | |
| Mutation | I/L | Q/V | V/W | N/L | S/L | G/GG |
| combination 29 | | | | | | |
| Mutation | I/V | N/V | V/F | N/I | S/V | G/N |
| combination 30 | | | | | | |
| Mutation | L/I | N/V | I/W | N/I | T/I | P/N |
| combination 31 | | | | | | |
| Mutation | L/L | Q/V | I/F | N/L | T/L | G/N |
| combination 32 | | | | | | |
| Mutation | L/V | N/L | L/W | N/V | T/V | G/GG |
| combination 33 | | | | | | |
| Mutation | V/I | Q/L | L/F | Q/I | Y/I | A/GG |
| combination 34 | | | | | | |
| Mutation | V/L | N/I | V/W | Q/L | Y/L | A/N |
| combination 35 | | | | | | |
| Mutation | V/V | Q/I | V/F | Q/V | Y/V | P/GG |
| combination 36 | | | | | | |
| Mutation | I/I | Q/I | I/W | Q/V | T/I | P/GG |
| combination 37 | | | | | | |
| Mutation | I/L | N/I | I/F | Q/L | T/L | A/N |
| combination 38 | | | | | | |
| Mutation | I/V | Q/L | L/W | Q/I | T/V | G/GG |
| combination 39 | | | | | | |
| Mutation | L/I | N/L | L/F | N/V | Y/I | G/N |
| combination 40 | | | | | | |
| Mutation | L/L | Q/V | V/W | N/L | Y/L | P/N |
| combination 41 | | | | | | |
| Mutation | V/I | N/V | I/W | N/I | Y/V | G/N |
| combination 42 | | | | | | |
| Mutation | V/I | N/V | I/W | N/I | S/I | G/GG |
| combination 43 | | | | | | |
| Mutation | V/L | Q/V | I/F | N/L | S/L | A/GG |
| combination 44 | | | | | | |
| Mutation | v/v | N/L | L/W | N/V | S/V | A/N |
| combination 45 | | | | | | |
| Mutation | I/I | Q/L | L/F | Q/I | S/I | P/GG |
| combination 46 | | | | | | |
| Mutation | I/L | N/I | V/W | Q/L | S/L | P/GG |
| combination 47 | | | | | | |
| Mutation | I/V | Q/I | V/F | Q/V | S/V | P/N |
| combination 48 | | | | | | |
| Mutation | L/I | Q/I | I/W | Q/V | T/I | P/GG |
| combination 49 | | | | | | |
| Mutation | L/L | N/I | I/F | Q/L | T/L | P/N |
| combination 50 | | | | | | |
| Mutation | L/V | Q/L | L/W | Q/I | T/V | A/GG |
| combination 51 | | | | | | |
| Mutation | V/I | N/L | L/F | N/V | Y/I | A/N |
| combination 52 | | | | | | |
| Mutation | V/L | Q/V | V/W | N/L | Y/L | G/GG |
| combination 53 | | | | | | |
| Mutation | V/V | N/V | V/F | N/I | Y/V | G/N. |
| combination 54 | | | | | | |

20. The isolated binding protein comprising an antigen-binding domain according to claim 1, wherein the binding protein is one of a nanobody, F(ab′)₂, Fab′, Fab, Fv, scFv, a bispecific antibody and a minimal recognition unit of antibody.

21. The isolated binding protein comprising an antigen-binding domain according to claim 1, wherein the binding protein comprises light chain framework regions FR-L1, FR-L2, FR-L3 and FR-L4 with sequences correspondingly shown in SEQ ID NO: 1-4, and/or, heavy chain framework regions FR-H1, FR-H2, FR-H3 and FR-H4 with sequences correspondingly shown in SEQ ID NO: 5-8.

22. The isolated binding protein comprising an antigen-binding domain according to claim 1, wherein the binding protein is labeled by an indicator which shows signal intensity.
